(19) United States
(12) Patent Application Publication
(10) Pub. No.: US 2024/0394876 A1
(43) Pub. Date: Nov. 28, 2024

(54) INFORMATION PROCESSING APPARATUS, BIOMETRIC INFORMATION ESTIMATION APPARATUS, INFORMATION PROCESSING METHOD, AND RECORDING MEDIUM

(71) Applicant: NEC Corporation, Minato-ku, Tokyo (JP)

(72) Inventors: Yusuke AKAMATSU, Tokyo (JP); Yoshifumi ONISHI, Tokyo (JP)

(73) Assignee: NEC Corporation, Minato-ku, Tokyo (JP)

(21) Appl. No.: 18/695,412

(22) PCT Filed: Sep. 30, 2021
(86) PCT No.: PCT/JP2021/036092

§ 371 Date: Mar. 26, 2024

(51) Int. Cl. G06T 7/00; A61B 5/0205; A61B 5/024; A61B 5/1455

(52) U.S. Cl. CPC G06T 7/0012; A61B 5/0205; A61B 5/14551; A61B 5/02416; G06T 2207/20081; G06T 2207/30004; G06T 2207/30201

(57) ABSTRACT

An information processing apparatus includes:
an acquisition unit that acquires a partial modal set that includes at least a first type of modal of multiple types of modals and that does not include a second type of modal of the multiple types of modals;
a modal output unit that allows a modal generation model to output at least the second type of modal, by inputting the partial modal set to the modal generation model, wherein the modal generation model outputs at least one of the multiple types of modals in a case where at least one of the multiple types of modals is inputted, and the modal generation model is generated by machine learning using a complete modal set including the multiple types of modals; and
a modal generation unit that generates a generation complete modal set including the partial modal set and the second type of modal outputted by the modal generation model.

### TECHNICAL FIELD

This disclosure relates to technical fields of an information processing apparatus, a biometric information estimation apparatus, an information processing method, and a recording medium.

### BACKGROUND ART

Patent Literature describes an example of an apparatus that detects heart rate and respiratory rate from a face image. Furthermore, Patent Literature 2 describes an example of a system that determines stress coping of a subject on the basis of the face image and some of the mean blood pressure, heart rate, cardiac output, and total peripheral resistance. In addition, Patent Literature describes an example of a function for re-training algorithm on the basis of new training data acquired. Furthermore, Patent Literature 4 describes a method of automatically performing re-training of a speech recognition system.

### CITATION LIST

### Patent Literature

Patent Literature 1: JP2012-239661A
Patent Literature 2: JP2021-037287A
Patent Literature 3: International Publication No. WO2021/075288
Patent Literature 4: JP2003-524805A

### SUMMARY

### Technical Problem

It is an example object of this disclosure to provide an information processing apparatus, a biometric information estimation apparatus, an information processing method, and a recording medium that are intended to improve the techniques/technologies described in Citation List.

### Solution to Problem

An information processing apparatus according to an example aspect of this disclosure includes: an acquisition unit that acquires a first modal set that includes at least a first type of modal of multiple types of modals and that does not include a second type of modal of the multiple types of modals; an output unit that allows a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and a modal generation unit that generates a third modal set including the first modal set and the second type of modal outputted by the modal generation model.

A biometric information estimation apparatus according to an example aspect of this disclosure includes: an image acquisition unit that acquires a face image of a target person; and a biometric information estimation unit that allows a modal estimation model to output biometric information on the target person as an output modal, wherein the modal estimation model outputs a fourth type of modal that is different from a third type of modal of the multiple types of modals as an output modal, in a case where the third type of modal of the multiple types of modals is inputted as an input modal, by performing machine learning using a third modal set including (i) a first modal set that includes at least a first type of modal of the multiple types of modals and that does not include a second type of modal of the multiple types of modals, and (ii) the second type of modal outputted by a modal generation model, wherein the modal generation model outputs at least one of the multiple types of modals in a case where at least one of the multiple types of modals is inputted, and the modal generation model is generated by machine learning using a second modal set including the multiple types of modals.

An information processing method according to an example aspect of this disclosure includes: acquiring a first modal set that includes at least a first type of modal of multiple types of modals and that does not include a second type of modal of the multiple types of modals; allowing a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and generating a third modal set including the first modal set and the second type of modal outputted by the modal generation model.

A recording medium according to a first aspect of this disclosure is a recording medium on which a computer program that allows a computer to execute an information processing method is recorded, the information processing method including: acquiring a first modal set that includes at least a first type of modal of multiple types of modals and that does not include a second type of modal of the multiple types of modals; allowing a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and generating a third modal set including the first modal set and the second type of modal outputted by the modal generation model.

### BRIEF DESCRIPTION OF DRAWINGS

FIG. 1 is a block diagram illustrating a configuration of an information processing apparatus according to a first example embodiment.

FIG. 2 is a block diagram illustrating a configuration of an information processing apparatus according to a second example embodiment.

FIG. 3A to FIG. 3C illustrate examples of data structures of data groups used in the second example embodiment.

FIG. 4 is a flowchart illustrating a flow of a modal set generation operation performed by the information processing apparatus according to the second example embodiment.

FIG. 5A to FIG. 5D are block diagrams illustrating configurations of a modal generation unit 212 included in an information processing apparatus according to a third example embodiment.

FIG. 6 is a block diagram illustrating a configuration of an information processing apparatus according to a fourth example embodiment.

FIG. 7 is a flowchart illustrating a flow of a modal estimation model generation operation performed by the information processing apparatus according to the fourth example embodiment.

FIG. 8 is a block diagram illustrating a configuration of an on-line diagnosis support system including a diagnosis support apparatus according to a fifth example embodiment.

FIG. 9 is a block diagram illustrating a configuration of a biometric information estimation unit included in the diagnosis support apparatus according to the fifth example embodiment.

FIG. 10 is a diagram illustrating a flow of an on-line diagnosis support operation performed by the on-line diagnosis support system including the diagnosis support apparatus according to the fifth example embodiment.

### DESCRIPTION OF EXAMPLE EMBODIMENTS

Hereinafter, with reference to the drawings, an information processing apparatus, a biometric information estimation apparatus, an information processing method, and a recording medium according to example embodiments will be described.

### (1) First Example Embodiment

First, an information processing apparatus, an information processing method, and a recording medium according to a first example embodiment will be described. The following describes the information processing apparatus, the information processing method, and the recording medium according to the first example embodiment, by using an information processing apparatus 1 to which the information processing apparatus, the information processing method, and the recording medium according to the first example embodiment are applied.

### (1-1) Configuration of Information Processing Apparatus 1

FIG. 1 is a block diagram illustrating a configuration of the information processing apparatus 1 according to the first example embodiment. As illustrated in FIG. 1, the information processing apparatus 1 includes an acquisition unit 11 and a modal generation unit 12.

First, a modal may refer to biometric information. A modal set may refer to data including one or more types of modals for a certain individual, for example. For example, it is assumed that data including three types of modals that are a modal A, a modal B, and a modal C for a certain individual are required. In this case, in the first example embodiment, the data including all the three types that are the modal A, the modal B, and the modal C, are referred to as a complete modal set. On the other hand, data including a part of the modal A, the modal B, and the modal C, are referred to as a partial modal set. The partial modal set may be any of the followings: data including the modal A and the modal B; data including the modal A and the modal C; data including the modal B and the modal C; data including the modal A; data including the modal B; and data including the modal C.

The acquisition unit 11 acquires a partial modal set IMS as a first modal set. The partial modal set IMS includes at least a first type of modal 1M of multiple types of modals, but does not include a second type of modal 2M of the multiple types of modals.

A modal output unit 13 allows a modal generation model to output at least the second type of modal, by inputting the partial modal set IMS to the modal generation model.

The modal generation unit 12 generates a generation complete modal set GMS, as a third modal set including the partial modal set IMS and the second type of modal outputted by the modal generation model. The generation complete modal set GMS is a complete modal set including all the types of modals.

The modal generation model outputs at least one of multiple types of modals in a case where at least one of the multiple types of modals is inputted. Furthermore, the modal generation model is a model generated by machine learning using a complete modal set MMS as a second modal set including all the types of modals.

### (1-2) Technical Effect of Information Processing Apparatus 1

In the information processing apparatus 1 according to the first example embodiment, the modal generation model outputs the modal in which at least the second type of modal, i.e., the partial modal set IMS, is not included. Thus, the modal generation unit 12 is allowed to generate the complete modal set including multiple types of modals. That is, the partial modal set that is relatively easy to collect and that does not include a part of types of modals, may be utilized to generate the complete modal set that is relatively difficult to collect and that includes multiple types of modals. Therefore, the information processing apparatus 1 according to the first example embodiment is configured to relatively easily acquire the complete modal set including a large number of multiple types of modals. Since the complete modal set including a large number of multiple types of modals may be utilized for machine learning of the model that realizes high-accuracy estimation, the complete modal set may contribute to realization of high-accuracy modal estimation.

### (2) Second Example Embodiment

Next, an information processing apparatus, an information processing method, and a recording medium according to a second example embodiment will be described. The following describes the information processing apparatus, the information processing method, and the recording medium according to the second example embodiment, by using an information processing apparatus 2 to which the information processing apparatus, the information processing method, and the recording medium according to the second example embodiment are applied.

### (2-1) Configuration of Information Processing Apparatus 2

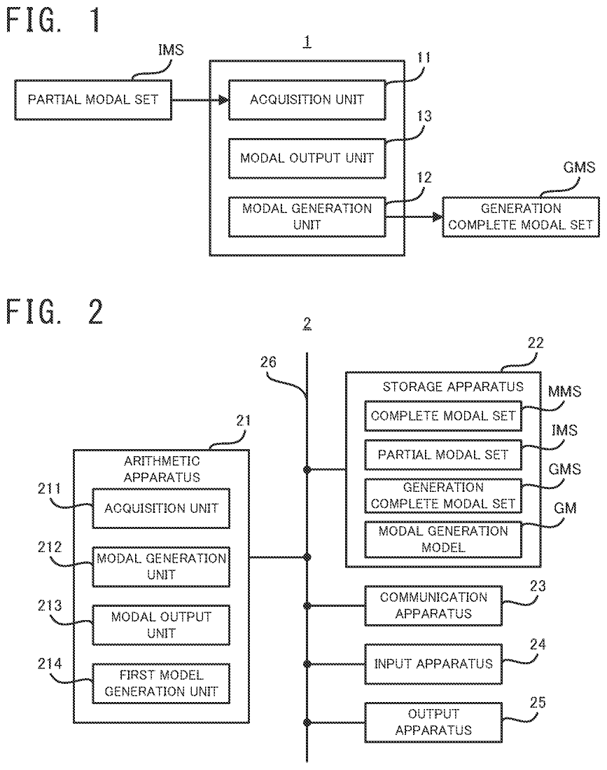

First, with reference to FIG. 2, a configuration of the information processing apparatus 2 according to the second example embodiment will be described. FIG. 2 is a block diagram illustrating the configuration of the information processing apparatus 2 according to the second example embodiment. In the following description, the components already described carry the same reference numerals, and a detailed description thereof will be omitted.

As illustrated in FIG. 2, the information processing apparatus 2 includes an arithmetic apparatus 21 and a storage apparatus 22. Furthermore, the information processing apparatus 2 may include a communication apparatus 23, an input apparatus 24, and an output apparatus 25. The information processing apparatus 2 may not include at least one of the communication apparatus 23, the input apparatus 24, and the output apparatus 25. The arithmetic apparatus 21, the storage apparatus 22, the communication apparatus 23, the input apparatus 24, and the output apparatus 25 may be connected through a data bus 26.

The arithmetic apparatus 21 includes at least one of a CPU (Central Processing Unit), a GPU (Graphics Processing Unit), and a FPGA (Field Programmable Gate Array), for example. The arithmetic apparatus 21 reads a computer program. For example, the arithmetic apparatus 21 may read a computer program stored in the storage apparatus 22. For example, the arithmetic apparatus 21 may read a computer program stored by a computer-readable and non-transitory recording medium, by using a not-illustrated recording medium reading apparatus provided in the information processing apparatus 2 (e.g., the input apparatus 24 described later). The arithmetic apparatus 21 may acquire (i.e., download or read) a computer program from a not-illustrated apparatus disposed outside the information processing apparatus 2, through the communication apparatus 23 (or another communication apparatus). The arithmetic apparatus 21 executes the read computer program. Consequently, a logical functional block for performing an operation to be performed by the information processing apparatus 2 is realized or implemented in the arithmetic apparatus 21. That is, the arithmetic apparatus 21 is allowed to function as a controller for realizing or implementing the logical functional block for performing an operation (in other words, a process) to be performed by the information processing apparatus 2.

FIG. 2 illustrates an example of the logical functional block realized or implemented in the arithmetic apparatus 21 to perform a modal generation operation. As illustrated in FIG. 2, an acquisition unit 211 that is a specific example of the “acquiring unit”, a modal generation unit 212 that is a specific example of the “modal generation unit”, a modal output unit 213 that is a specific example of the “output unit”, and a first model generation unit 214 that is a specific example of the “first model generation unit” are realized or implemented in the arithmetic apparatus 21. The first model generation unit 214 generates a modal generation model GM by performing machine learning using the complete modal set MMS as the second modal set.

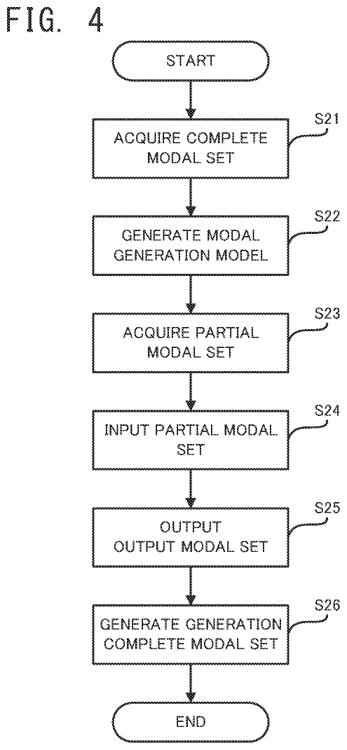

Details of operation of each of the acquisition unit 211, the modal generation unit 212, the modal output unit 213, and the first model generator 214 will be described in detail later with reference to FIG. 4. The arithmetic apparatus 21 may not include the first model generation unit 214.

The storage apparatus 22 is configured to store desired data. For example, the storage apparatus 22 may temporarily store a computer program to be executed by the arithmetic apparatus 21. The storage apparatus 22 may temporarily store data that are temporarily used by the arithmetic apparatus 21 when the arithmetic apparatus 21 executes the computer program. The storage apparatus 22 may store data that are stored by the information processing apparatus 2 for a long time. The storage apparatus 22 may include at least one of a RAM (Random Access Memory), a ROM (Read Only Memory), a hard disk apparatus, a magneto-optical disk apparatus, a SSD (Solid State Drive), and a disk array apparatus. That is, the storage apparatus 22 may include a non-transitory recording medium.

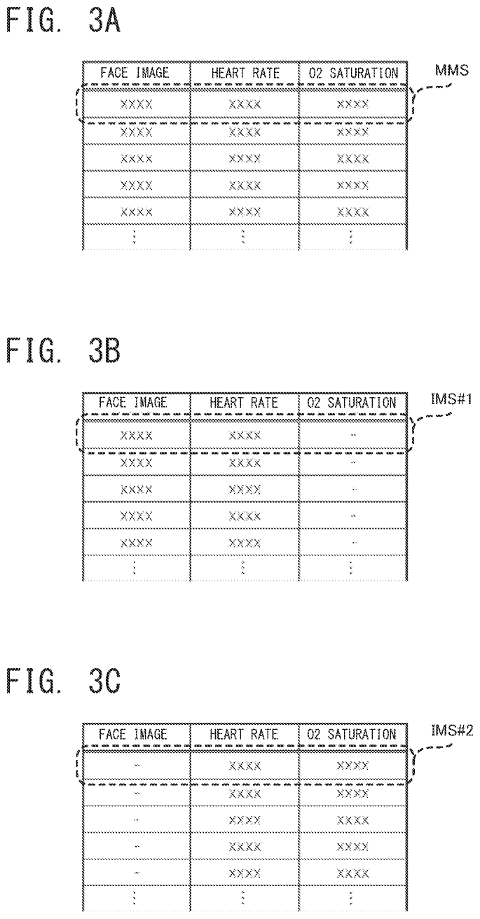

The storage apparatus 22 may store the complete modal set MMS, the partial modal set IMS, the generation complete modal set GMS, and the modal generation model GM. Details of the complete modal set MMS and the partial modal set IMS will be described in detail later with reference to FIG. 3A to FIG. 3C. The storage apparatus 22 may not store the complete modal set MMS, the partial modal set IMS, the generation complete modal set GMS, and the modal generation model GM. Here, the complete modal set MMS is a modal set including all the types of modals.

The communication apparatus 23 is configured to communicate with an apparatus external to the information processing apparatus 2, through a not-illustrated communication network.

The input apparatus 24 is an apparatus that receives an input of information to the information processing apparatus 2 from an outside of the information processing apparatus 2. For example, the input apparatus 24 may include an operating apparatus (e.g., at least one of a keyboard, a mouse, and a touch panel) that is operable by an operator of the information processing apparatus 2. For example, the input apparatus 24 may include a reading apparatus that is configure to read information recorded as data on a recording medium that is externally attachable to the information processing apparatus 2.

The output apparatus 25 is an apparatus that outputs information to the outside of the information processing apparatus 2. For example, the output apparatus 25 may output the information as an image. That is, the output apparatus 25 may include a display apparatus (a so-called display) that is configured to display an image indicating the information that is desirably outputted. For example, the output apparatus 25 may output information as audio. That is, the output apparatus 25 may include an audio apparatus (a so-called speaker) that is configured to output audio. For example, the output apparatus 25 may output information onto a paper surface. That is, the output apparatus 25 may include a print apparatus (a so-called printer) that is configured to print desired information on the paper surface.

There is a high demand for estimating the modal with high accuracy by the model generated by machine learning. In order to generate the model that realizes high-accuracy estimation by machine learning, a training data group including a large amount of training data is required. As the training data, it is preferable to use the complete modal set including required multiple types of modals (e.g., three types that are a face image, heart rate, and oxygen saturation). It is, however, generally hard to collect the training data group including a large number of complete modal sets of required multiple types of modals (e.g., three types that are a face image, heart rate, and oxygen saturation). In addition, as the number of types of modals that is an estimation target increases, the number of types of modals to be included increases, and therefore, a cost of collection of the training data increases. On the other hand, it is easier to collect the partial modal set that lacks a part of types of modals, than to collect the complete modal set including all the types of modals. For example, the complete modal set including all the three types of modals that are a face image, heart rate, and oxygen saturation for a certain individual, can be counted as one sample, and a large number of complete modal sets may refer to a complete modal set group including thousands to 10000 samples, or more, for example.

In the second example embodiment, multiple types of modals may include a face image, heart rate, and oxygen saturation, for example. FIG. 3A to FIG. 3C respectively illustrate examples of data structures of data groups used in the second example embodiment.

FIG. 3A illustrates the data structure of a training data group used by the first model generation unit 214 for machine learning of the modal generation model GM. The training data group may include 2000 complete modal sets MMS, for example. In the second example embodiment, the complete modal set MMS may include a face image, heart rate, and oxygen saturation. In the second example embodiment, each modal included in the complete modal set MMS may be data obtained by actual measurement.

FIG. 3B illustrates the data structure of a first input data group inputted by the modal output unit 213 to the modal generation model GM. The first input data group may include 4000 first partial modal sets IMS #1, for example. The partial modal set IMS includes at least the first type of modal 1M of multiple types of modals, but does not include the second type of modal 2M of the multiple types of modals. In the second example embodiment, the first type of modal 1M in the first partial modal set IMS #1 may be a face image and heart rate, and the first partial modal set IMS #1 may include a face image and heart rate. Furthermore, the second type of modal 2M may be oxygen saturation. The face image and the heart rate are often acquired simultaneously, and it is thus relatively easy to acquire a large number of partial modal sets including a combination of the face image and the heart rate.

FIG. 3C illustrates the data structure of a second input data group inputted by the modal output unit 213 to the modal generation model GM. The second input data group may include 4000 second partial modal sets IMS #2, for example. In the second example embodiment, the first type of modal 1M in the second partial modal set IMS #2 may be heart rate and oxygen saturation, and the second partial modal set may include heart rate and oxygen saturation. The second type of modal 2M may be a face image. Compared with the face image, data on heart rate and oxygen saturation have a smaller data volume. Furthermore, the face image is hard to collect from a privacy point of view, while it is relatively easy to collect the heart rate and the oxygen saturation. Therefore, it is relatively easy to acquire a large number of partial modal sets including a combination of the heart rate and the oxygen saturation.

In the second example embodiment, each modal included in the partial modal set IMS may be data obtained by actual measurement.

The modal generation unit 212 is configured to input the first input data group and the second input data group to the modal generation model GM and to generate a data group including 8000 generation complete modal sets GMS. The generation complete modal set GMS is a modal set including multiple types of modals, i.e., including the partial modal set IMS and the second type of modal 2M outputted by the modal generation model GM. Therefore, the data structure of the generation complete modal set GMS is the same as the data structure illustrated in FIG. 3A. That is, in the above example, it is possible to collect a training data group including 10000 complete modal sets including multiple types of modals, i.e., 2000 complete modal sets MMS and 8000 generation modal sets GMS.

### (2-2) Modal Set Generation Operation Performed by Information Processing Apparatus 2

Next, with reference to FIG. 4, a modal set generation operation performed by the information processing apparatus 2 according to the second example embodiment will be described. FIG. 4 is a flowchart illustrating a flow of the modal set generation operation performed by the information processing apparatus 2 according to the second example embodiment.

As illustrated in FIG. 4, the acquisition unit 211 acquires the complete modal set MMS (step S21). In the second example embodiment, the complete modal set MMS may include a face image, heart rate, and oxygen saturation, and there may be, for example, 2000 complete modal sets. The first model generation unit 214 generates the modal generation model GM by performing machine learning using the complete modal set MMS acquired in the step S21 (step S22).

The modal generation model GM is a model that is configured to output at least one of multiple types of modals in a case where at least one of the multiple types of modals is inputted. In a case where the complete modal set GM used to generate the modal generation model MMS includes a face image, heart rate, and oxygen saturation as described above, the first model generation unit 214 may generate the modal generation model GM that is configured to output at least one of the face image, heart rate, and oxygen saturation in a case where at least one of the face image, heart rate, and oxygen saturation is inputted by machine learning. Specifically, the first model generation unit 214 may generate the modal generation model GM by adjusting a parameter of the modal generation model GM so as to reduce (preferably minimize) a value of a loss function that is set on the basis of at least one of an error between the face image outputted by the modal generation model GM and the face image included in the complete modal set MMS, an error between the heart rate outputted by the modal generation model GM and the heart rate included in the complete modal set MMS, and an error between the oxygen saturation outputted by the modal generation model GM and the oxygen saturation included in the complete modal set MMS.

Next, the acquisition unit 211 acquires the partial modal set IMS (step S23). In the second example embodiment, the partial modal set IMS may include, for example, 4000 first partial modal sets IMS #1 including a face image and heart rate, and 4000 second partial modal sets IMS #2 including heart rate and oxygen saturation. The modal output unit 213 inputs the partial modal set IMS to the modal generation model GM (step S24). The modal output unit 213 allows the modal generation model GM to output at least the second type of modal 2M (step S25). The modal generation model GM may output the oxygen saturation for an input of the partial modal set IMS #1 and may output information indicating the face image for an input of the partial modal set IMS #2. The modal generation unit 212 generates, for example, 8000 complete modal sets GMS including the partial modal set IMS and the second type of modal 2M outputted by the modal generation model GM (step S26).

The modal generation model GM may output at least one of the face image and the heart rate, as well as the oxygen saturation as the second type of modal 2M, in a case where the face image and the heart rate are inputted as the partial modal set IMS. In this situation, for at least one of the face image and the heart rate, there are two types of modals that are the modal inputted to the modal generation model GM and the modal outputted from the modal generation model GM. The second example embodiment describes a case where the modal generation unit 212 generates the generation complete modal set GMS including the partial modal set IMS and the second type of modal 2M, but the modal generation unit 212 may generate the generation complete modal set GMS including the modal outputted from the modal generation model GM and the type of modal that is included in the partial modal set GM and that is not outputted by the modal generation model GM. For example, in a case where the first type of modal 1M is a face image and heart rate and the second type of modal 2M is oxygen saturation, and in a case where the modal generation model GM outputs the face image and the oxygen saturation, the modal generation unit 212 may generate the generation complete modal set GMS including the face image and the oxygen saturation outputted by the modal generation model GM and the heart rate included in the partial modal set IMS (the heart rate that is the type of modal that is included in the partial modal set IMS and that is not outputted by the modal generation model GM). In addition, the modal generation unit 212 may acquire a synthetic face image obtained by combining the face image included in the partial modal set IMS and the face image outputted by the modal generation model GM, and may generate the generation complete modal set GMS including the synthetic face image, the heart rate included in the partial modal set IMS, and the oxygen saturation outputted from the modal generation model GM. Consequently, the number of the generation complete modal sets GMS that may be generated by using the modal generation model GM is increased.

The modal included in the partial modal set IMS may be reliable data obtained by actual measurement, and the modal generation unit 212 may be considered to preferably generate the complete modal set GMS by using the modal included in the partial modal set IMS. On the other hand, there is a possibility that noise is excluded from the modal outputted from the modal generation model GM, and the modal generation unit 212 may be considered to preferably generate the generation complete modal set GMS by using the second type of modal 2M outputted from the modal generation model GM.

Alternatively, in the second example embodiment, it is intended to collect a large number of modal sets. Thus, three types of generation complete modal sets GMS that are a first generation complete modal set GMS using the modal included in the partial modal set IMS, a second generation complete modal set GMS using the modal outputted from the modal generation model GM, and a third generation complete modal set GMS using a synthetic modal obtained by combining the above two may be generated.

Specifically, for example, let us assume that the modal generation model GM outputs an oxygen saturation OC as the second type of modal 2M and outputs a face image OA and a heart rate OB in a case where a face image IA and a heart rate IB are inputted as the partial modal setting IMS. Here, a modal obtained by combining the face image IA and the face image OA is referred to as a face image CA, and a modal obtained by combining the heart rate IB and the heart rate OB is referred to as a heart rate CB. In this instance, the modal generation unit 212 is capable of generating nine types of generation complete modal sets GMS that are: a generation complete modal set GMS1 including the face image IA, the heart rate IB, and the oxygen saturation OC; a generation complete modal set GMS2 including the face image IA, the heart rate OB, and the oxygen saturation OC; a generation complete modal set GMS3 including the face image OA, the heart rate IB, and the oxygen saturation OC; a generation complete modal set GMS4 including the face image OA, the heart rate OB, and the oxygen saturation OC; a generation complete modal set GMS5 including the face image CA, the heart rate CB, and the oxygen saturation OC; a generation complete modal set GMS6 including the face image CA, the heart rate IB, and the oxygen saturation OC; a generation complete modal set GMS7 including the face image CA, the heart rate OB, and the oxygen saturation OC; a generation complete modal set GMS8 including the face image IA, the heart rate CB, and the oxygen saturation OC; and a generation complete modal set GMS9 including the face image OA, the heart rate CB, and the oxygen saturation OC.

The second example embodiment describes an example in which the face image and the heart rate are inputted to and at least the oxygen saturation is outputted from the modal generation model GM, and an example in which the heart rate and the oxygen saturation are inputted to and at least the face image is outputted from the modal generation model GM, but the input/output of the modal to/from the modal generation model GM is not limited to these two types. For example, the face image and the oxygen saturation may be inputted to and at least the heart rate may be outputted from the modal generation model GM. The face image may inputted to and at least the heart rate and the oxygen saturation may be outputted from the modal generation model GM. The heart rate may inputted to and at least the face image and the oxygen saturation may be outputted from the modal generation model GM. The oxygen saturation may inputted to and at least the face image and the heart rate may be outputted from the modal generation model GM.

### (2-3) Technical Effect of Information Processing Apparatus 2

The information processing apparatus 2 according to the second example embodiment is configured to estimate a modal other than the inputted modal with a certain degree of accuracy and to generate the modal generation model to be outputted, by performing machine learning using a relatively small number of complete modal sets MMS. Then, the information processing apparatus 2 according to the second example embodiment is configured to acquire a type of modal that is not included in the partial modal set IMS, by the modal generation model. That is, even if it is hard to collect a large number of complete modal sets MMS, but in a case where it is possible to collect a large number of partial modal sets IMS, the information processing apparatus 2 according to the second example embodiment is allowed to generate a large number of generation complete modal sets GMS including multiple types of modals, by using this acquired modal.

For example, let us assume that the complete modal set including three types of modals that are a face image, heart rate, and oxygen saturation, are required. Even if it is hard to collect a large number of complete modal sets, there may be case where it is possible to collect a large number of partial modal sets, such as a partial modal set including heart rate and oxygen saturation, and a partial modal set including a face image and heart rate. In such a case, the information processing apparatus 2 according to the second example embodiment is allowed to acquire a type of modal that is not included in the partial modal set, by the modal generation model, and to generate a large number of complete modal sets including three types of modals that are a face image, heart rate, and oxygen saturation.

Since this complete modal set including a large number of multiple types of modals can be utilized for machine learning of the model that realizes high-accuracy estimation, the complete modal set may contribute to realization of high-accuracy modal estimation.

Furthermore, when the number of types of modals to be estimated increases, it is harder to collect the training data in many cases. For example, in many cases, it is harder to collect a complete modal set including four types of modals (A, B, C, D), than to collect a complete modal set including three types of modals (A, B, C). The information processing apparatus 2 in the second example embodiment is configured to generate and acquire the increased type of modal D by using the generated modal generation model GM, and it is thus possible to relatively easily collect the complete modal set including the four types of modals (A, B, C, D). Therefore, the information processing apparatus 2 according to the second example embodiment is configured to relatively easily collect a large number of complete modal sets even when the number of types of modals increases. Since the large number of complete modal sets can be utilized for machine learning of the model that realizes high-accuracy estimation, and thus, the complete modal set may contribute to realization of high-accuracy estimation of various types of modals.

### (3) Third Example Embodiment

Next, an information processing apparatus, an information processing method, and a recording medium according to a third example embodiment will be described. The following describes the information processing apparatus, the information processing method, and the recording medium according to the third example embodiment, by using an information processing apparatus to which the information processing apparatus, the information processing method, and the recording medium according to the third example embodiment are applied.

The information processing apparatus according to the third example embodiment may have the same configuration as that of the information processing apparatus 2 according to the second example embodiment. The information processing apparatus according to the third example embodiment is different from the information processing apparatus 2 according to the second example embodiment in information inputted by the modal output unit 213 to the modal generation model GM. Other features of the information processing apparatus may be the same as those of the information processing apparatus 2.

### (3-1) Configuration of Modal Generation Unit 212

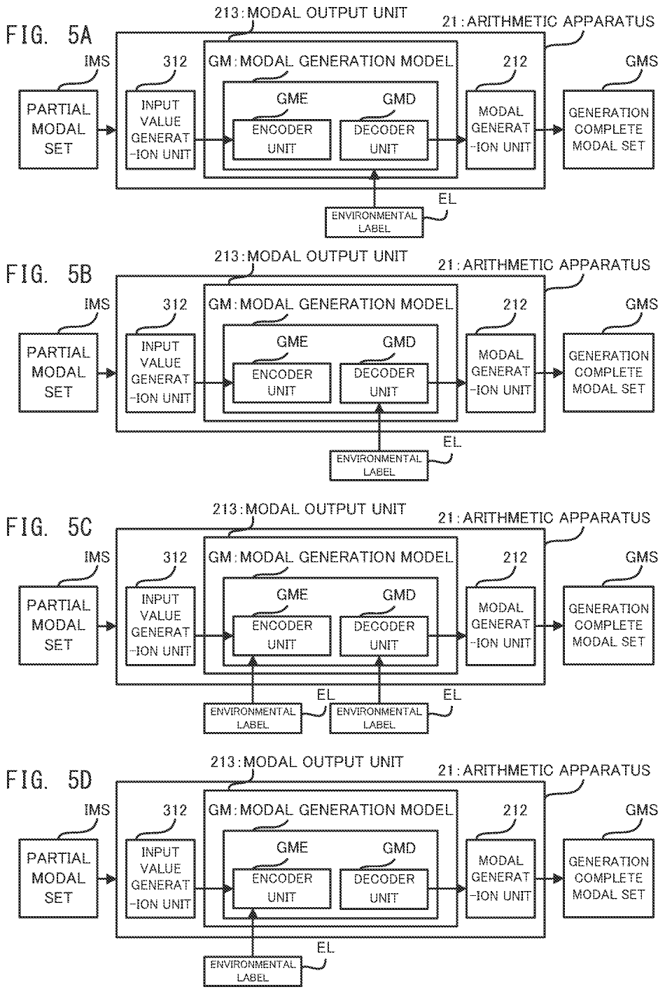

FIG. 5A is a block diagram illustrating a configuration of the modal generation unit 212. The modal generation unit 212 may include at least an input value generation unit 312 and the modal generation model GM. In a case where at least one of multiple types of modals is inputted, the input value generation unit 312 may generate an input value of at least one of the multiple types of modals. The input of at least one of the multiple types of modals to the modal generation model GM may be an input of an input value of at least one of the multiple types of modals. The input value may be, for example, features of the modal. In this case, the input value generation unit 312 may be any mechanism that is configured to extract the features of the modal, such as a mechanism utilizing a neural network.

The modal generation model GM may include at least an encoder unit GME and a decoder unit GMD. In a case where at least one of the multiple types of modals is inputted, the encoder unit GME may transform an input value of at least one of the multiple types of modals into latent variables. The decoder unit GMD may generate at least one of the multiple types of modals by reconstructing the latent variables.

The modal output unit 213 according to the third example embodiment may allow the modal generation model GM to output the second type of modal IMS, by inputting, to the modal generation model GM, the partial modal set IMS and an environmental label EL indicating an acquisition environment when the first type of modal 1M included in the partial modal set is acquired. By the modal output unit 213 inputting the environmental label EL, it is possible to prevent an adverse effect of the acquisition environment of the first type of modal 1M on the estimation of the second type of modal 2M by the modal generation model GM. The modal generation unit 212 generates the generation complete modal set GMS including the partial modal set IMS and the second type of modal 2M outputted by the modal generation model GM.

As illustrated in FIG. 5A, the modal output unit 213 may input the environmental label EL to at least one of the encoder unit GME and the decoder unit GMD. That is, as illustrated in FIG. 5B, the modal output unit 213 may input the environmental label EL to the decoder unit GMD. Furthermore, as illustrated in FIG. 5C, the modal output unit 213 may input the environmental label EL to both the encoder unit GME and the decoder unit GMD. In addition, as illustrated in FIG. 5D, the modal output unit 213 may input the environmental label EL to the encoder unit GME.

The environmental label EL is preferably information for providing information that contributes to estimation accuracy. An example of the environmental label EL may include information that is known at the time of modal acquisition, such as fixed information on equipment that acquires the modal, and target person information. For example, it may include information such as properties of a camera that captures a face image, a lighting condition, gender, age, and skin color of a target person, and type of operation (face direction, etc.) at the time of modal acquisition.

### (3-2) Technical Effect of Information Processing Apparatus 3

Since the information processing apparatus 3 according to the third example embodiment inputs the partial modal setting IMS and environmental information explicitly indicating the acquisition environment in the case where the inputted modal is acquired, the modal generation model GM may estimate the second type of modal 2M with higher accuracy than in a case where the environmental information is not inputted. Therefore, the information processing apparatus 3 according to the third example embodiment is capable of generating the complete modal set GMS that is significantly close to a real one and that includes the second type of modal 2M that is accurately estimated. Since the modal output unit 213 inputs the environmental information to at least one of the encoder unit GME and the decoder unit GMD, it is possible to control the estimation accuracy of the second type of modal 2M to be outputted. Then, the information processing apparatus 3 according to the third example embodiment is capable of controlling realness of the generation complete modal set GMS to be generated. When the modal output unit 213 inputs the environmental information to the encoder unit, the effect of the environmental information that contributes to the estimation accuracy may be reduced because of a transformation process by the encoder unit. Thus, by the modal output unit 213 inputting the environmental information to the decoder unit, it is expectedly possible to avoid a reduced effect of the environmental information that contributes to the estimation accuracy. The complete modal set GMS that is significantly close to a real one, can be utilized for machine learning of model that realizes high-accuracy estimation, and may contribute to realization of high-accuracy estimation of the modal.

### (4) Fourth Example Embodiment

Next, an information processing apparatus, an information processing method, and a recording medium according to a fourth example embodiment will be described. The following describes the information processing apparatus, the information processing method, and the recording medium according to the fourth example embodiment, by using an information processing apparatus 4 to which the information processing apparatus, the information processing method, and the recording medium according to the fourth example embodiment are applied.

### (4-1) Configuration of Information Processing Apparatus 4

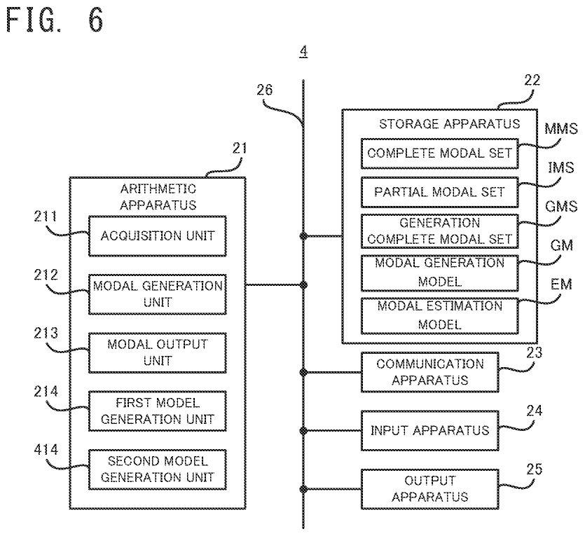

Hereinafter, a configuration of the information processing apparatus 4 according to the fourth example embodiment will be described with reference to FIG. 6. FIG. 6 is a block diagram illustrating the configuration of the information processing apparatus 4 according to the fourth example embodiment. In the following description, the components already described carry the same reference numerals, and a detailed description thereof will be omitted.

As illustrated in FIG. 6, the information processing apparatus 4 according to the fourth example embodiment is different from the information processing apparatus 2 according to the second example embodiment (or from the information processing apparatus according to the third example embodiment) in that the arithmetic apparatus 21 includes a second model generation unit 414. Other features of the information processing apparatus 4 may be the same as those of the information processing apparatus 2 (or the information processing apparatus according to the third example embodiment).

The second model generation unit 414 is configured to generate a modal estimation model EM that outputs, in a case where a third type of modal 3M that is one type of multiple types of modals is inputted as an input modal, a fourth type of modal 4M that is different from the one type of multiple types of modal as an output modal, by performing machine learning using the generation complete modal set GMS. The storage apparatus 22 may store the generated modal estimation model EM. The modal estimation model EM generated in the fourth example embodiment may be, for example, a trained model that outputs heart rate and oxygen saturation in a case where a face image is inputted.

### (4-2) Modal Estimation Model EM Generation Operation Performed by Information Processing Apparatus 4

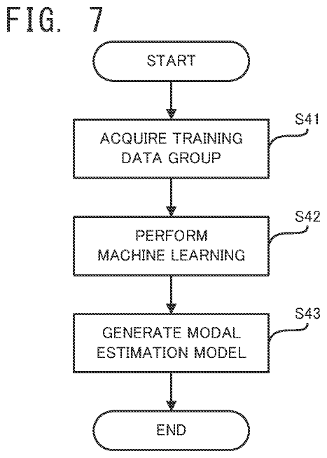

Next, with reference to FIG. 7, a modal estimation model EM generation operation performed by the information processing apparatus 4 according to the fourth example embodiment will be described. FIG. 7 is a flowchart illustrating a flow of the modal estimation model EM generation operation performed by the information processing apparatus 4 according to the fourth example embodiment.

As illustrated in FIG. 7, the acquisition unit 211 acquires a training data group (S41 of steps). The training data group is a complete modal set including multiple types of modals, and may include the complete modal set MMS and the generation complete modal set GMS. The second model generation unit 414 generates the modal estimation model EM by performing machine learning using the training data group acquired in the step S41 (step S42).

In the fourth example embodiment, the first model generation unit 214 and the second model generation unit 414 are described as separate configurations, and the modal generation model GM and the modal estimation model EM are described as separate models; however, the first model generation unit 214 and the second model generation unit 414 may have the same configuration, and the modal generation model GM and the modal estimation model EM may be the same model. That is, a control mechanism (i.e., the first model generation unit 214 and the second model generation unit 414) may generate a trained model (i.e., the modal generation model GM and the modal estimation model EM) by performing machine learning using the complete modal set MMS and may allow re-training of the trained model by performing machine learning using the generation complete modal set GMS, thereby to complete the trained model that allows high-accurate estimation.

The modal that can be estimated, may also include not only the heart rate and the oxygen saturation, but also respiratory rate, stress level, blood pressure level, cardiac output, total peripheral resistance, pulse, electrocardiogram, body temperature, body moisture status, alcohol concentration, lactic acid level, blood sugar level, muscle activity, line-of-sight movement, brain activity, consciousness level, and the like, for example. In addition, the modal that can be estimated, may be other than the heart rate and the oxygen saturation: for example, respiratory rate, stress level, blood pressure level, cardiac output, total peripheral resistance, pulse, electrocardiogram, body temperature, body moisture status, alcohol concentration, lactic acid level, blood sugar level, muscle activity, line-of-sight movement, brain activity, consciousness level, and the like.

It is also expected that modal estimation accuracy is higher for a target person for whom a modal set used for machine learning is collected, than for a target person for whom the modal set used for machine learning is not collected (hereinafter referred to as a “new target person”). Thus, in a case where the modal for a new target person is estimated, a corresponding modal set for the new target person may be collected in advance, and the modal estimation model EM may be re-trained. In addition, the modal set for the new target person that lacks a part of types of modals may be used to generate the modal set including all the modals and then to re-train the modal estimation model EM. This makes it possible to obtain the model that allows high-accuracy estimation of the modal for the new target person.

### (4-3) Technical Effect of Information Processing Apparatus 4

It is Expected that the Model Generated by Machine Learning May Accurately Estimate the modal even when there is an environmental change, such as a change in an imaging environment and movement of the target person. It is also expected that machine learning using a large amount of training data may generate the model that allows realization of higher-accuracy estimation, than in a case of machine learning using a small amount of training data.

Since the information processing apparatus 4 according to the fourth example embodiment generates the modal generation model by using a large amount of training data, it is possible to obtain the modal estimation model EM that allows higher-accuracy estimation, thereby realizing higher-accuracy estimation of the modal.

### (5) Fifth Example Embodiment

Next, a biometric information estimation apparatus according to a fifth example embodiment will be described. Hereinafter, the biometric information estimation apparatus according to the fifth example embodiment will be described by using an on-line diagnosis support system 500 including a diagnosis support apparatus 50 to which he biometric information estimation apparatus according to the fifth example embodiment is applied.

### (5-1) Configuration of On-Line Diagnosis Support System 500

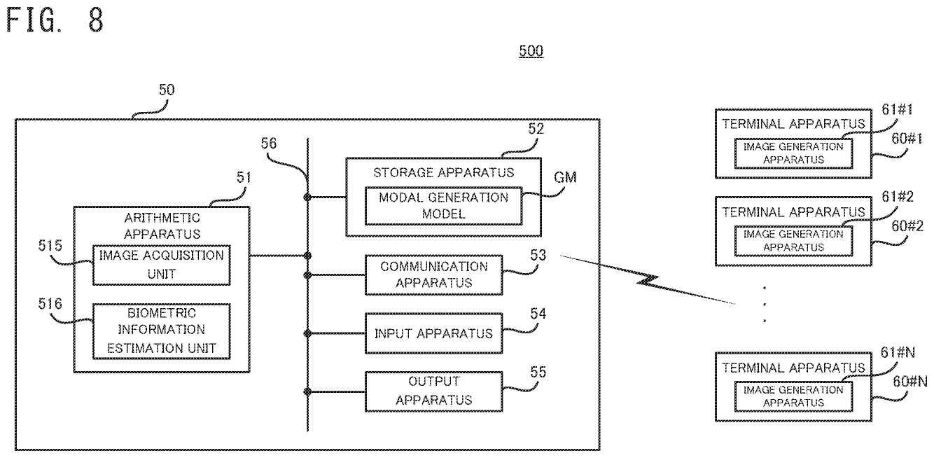

First, a configuration of the on-line diagnosis support system 500 according to the fifth example embodiment will be described with reference to FIG. 8. FIG. 8 is a block diagram illustrating the configuration of the on-line diagnosis support system 500 according to the fifth example embodiment. As illustrated in FIG. 8, the on-line diagnosis support system 500 includes the diagnosis support apparatus 50 and a plurality of terminal apparatuses 60 #1, 60 #2, . . . , and 60 #N (simply referred to as “60” when each of them is not distinguished).

The diagnosis support apparatus 50 may be, for example, an apparatus used by a doctor in diagnosis. The terminal apparatus 60 may also be, for example, an apparatus for use by a patient in a remote location. The terminal apparatus 60 may be equipped with an image generation apparatus 61 that generates a face image by imaging a target person.

### (5-2) Configuration of Diagnosis Support Apparatus 50

As illustrated in FIG. 8, the diagnosis support apparatus 50 includes an arithmetic apparatus 51 and a storage apparatus 52. In addition, the diagnosis support apparatus 50 may include a communications apparatus 53, an input apparatus 54, and an output apparatus 55. The diagnosis support apparatus 50, however, may not include at least one of the communication apparatus 53, the input apparatus 54, and the output apparatus 55. The arithmetic apparatus 51, the storage apparatus 52, the communication apparatus 53, the input apparatus 54, and the output apparatus 55 may be connected through a data bus 56.

The arithmetic apparatus 51 includes at least one of a CPU (Central Processing Unit), a GPU (Graphics Processing Unit), and a FPGA (Field Programmable Gate Array), for example. The arithmetic apparatus 51 reads a computer program. For example, the arithmetic apparatus 51 may read a computer program stored in the storage apparatus 52. For example, the arithmetic apparatus 51 may read a computer program stored by a computer-readable and non-transitory recording medium, by using a not-illustrated recording medium reading apparatus provided in the diagnosis support apparatus 50 (e.g., the input apparatus 54 described later). The arithmetic apparatus 51 may acquire (i.e., download or read) a computer program from a not-illustrated apparatus disposed outside the diagnosis support apparatus 50, through a communication apparatus 53 (or another communication apparatus). The arithmetic apparatus 51 executes the read computer program. Consequently, a logical functional block for performing an operation to be performed by the diagnosis support apparatus 50 is realized or implemented in the arithmetic apparatus 51. That is, the arithmetic apparatus 51 is allowed to function as a controller for realizing or implementing the logical functional block for performing an operation (in other words, a process) to be performed by the diagnosis support apparatus 50.

FIG. 8 illustrates an example of the logical functional block realized or implemented in the arithmetic apparatus 51 to perform a biometric information estimation operation. As illustrated in FIG. 8, an image acquisition unit 515 that is a specific example of the “image acquisition unit”, and a biometric information estimation unit 516 that is a specific example of the “biometric information estimation unit” are realized or implemented in the arithmetic apparatus 51.

### (5-3) Estimation Operation Using Modal Estimation Model EM

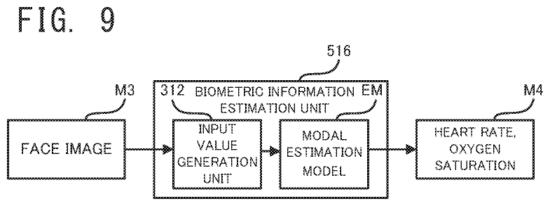

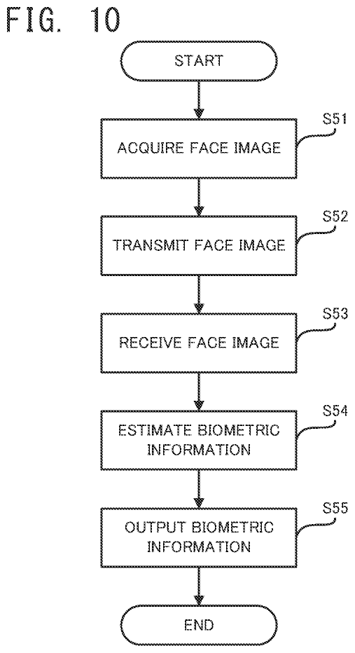

As illustrated in FIG. 9, the biometric information estimation unit 516 may include at least the input value generation unit 312 and the modal estimation model EM. The input value generation unit 312 may generate an input value of a face image in a case where the face image is inputted as an input modal. The input value generation unit 312 may input the input value of the face image to the modal estimation model EM, and the biometric information estimation unit 516 may allow the modal estimation model EM to output biometric information on the target person as an output modal.

Details of operation of each of the image acquisition unit 515 and the biometric information estimation unit 516 will be described in detail later with reference to FIG. 10.

The storage apparatus 52 is configured to store desired data. For example, the storage apparatus 52 may temporarily store a computer program to be executed by the arithmetic apparatus 51. The storage apparatus 52 may temporarily store data that are temporarily used by the arithmetic apparatus 51 when the arithmetic apparatus 51 executes the computer program. The storage apparatus 52 may store data that are stored by the diagnosis support apparatus 50 for a long time. The storage apparatus 52 may include at least one of a RAM (Random Access Memory), a ROM (Read Only Memory), a hard disk apparatus, a magneto-optical disk apparatus, a SSD (Solid State Drive), and a disk array apparatus. That is, the storage apparatus 52 may include a non-transitory recording medium.

The storage apparatus 52 may store the modal estimation model EM. The storage apparatus 52, however, may not store the modal estimation model EM.

The communication apparatus 53 is configured to communicate with an apparatus external to the diagnosis support apparatus 50, through a not-illustrated communication network. The diagnosis support apparatus 50 may be connected to the terminal apparatus 60 through the communication apparatus 53.

The input apparatus 54 is an apparatus that receives an input of information to the diagnosis support apparatus 50 from the outside of the diagnosis support apparatus 50. For example, the input apparatus 54 may include an operating apparatus (e.g., at least one of a keyboard, a mouse, and a touch panel) that is operable by an operator of the diagnosis support apparatus 50. For example, the input apparatus 54 may include a reading apparatus that is configured to read information recorded as data on a recording medium that is externally attachable to the diagnosis support apparatus 50.

The output apparatus 55 is an apparatus that outputs information to the outside of the diagnosis support apparatus 50. For example, the output apparatus 55 may output information as an image. That is, the output apparatus 55 may include a display apparatus (a so-called display) that is configured to display an image indicating the information that is desirably outputted. For example, the output apparatus 55 may output information as audio. That is, the output apparatus 55 may include an audio apparatus (a so-called speaker) that is configured to output audio. For example, the output apparatus 55 may output information onto a paper surface. That is, the output apparatus 55 may include a print apparatus (a so-called printer) that is configured to print desired information on the paper surface.

### (5-2) On-Line Diagnosis Support Operation Performed by On-Line Diagnosis Support System 500

Next, with reference to FIG. 10, an on-line diagnosis support operation performed by the on-line diagnosis support system 500 according to the fifth example embodiment will be described. FIG. 10 is a flowchart illustrating a flow of the on-line diagnosis support operation performed by the on-line diagnosis support system 500 according to the fifth example embodiment.

As illustrated in FIG. 10, the image generation apparatus 61 acquires a face image of a patient as the target person (step S51), and the terminal apparatus 60 transmits the face image to the diagnosis support apparatus 50.

Subsequently, the image acquisition unit 515 acquires the face image of the target person from the terminal apparatus 60 through a communication line (step S53). The biometric information estimation unit 516 allows the modal estimation model EM to estimate the biometric information on the target person as the third type of modal 4M, by inputting the face image in the modal estimation model EM as the third type of modal 3M (step S54). As the modal estimation model EM, for example, a model generated by the information processing apparatus 4 according to the fourth example embodiment may be used. In addition, the biometric information may include information on at least one of the heart rate and the oxygen saturation.

Subsequently, for example, the output apparatus 55 notifies a user of the diagnosis support apparatus 50 of the estimated biometric information (step S55).

### (5-4) Technical Effect of Diagnosis Support Apparatus 50

For example, a patient with an infection should be properly diagnosed by a doctor, but it is not preferable that the patient is in contact with a patient, a health care professional, or the like in a medical facility. Due to this situation, there is a high demand for on-line diagnosis in which the patient who should be properly diagnosed by the doctor can be diagnosed at home without direct contact with those who are in the medical facility, including the doctor.

Since the diagnosis support apparatus 50 according to the fifth example embodiment is configured to estimate the biometric information from the face image, the doctor can receive a plurality of pieces of biometric information, such as heart rate and oxygen saturation, simply by acquiring the face image transmitted by the patient, for example. Therefore, the diagnosis support apparatus 50 according to the fifth example embodiment is capable of assisting the doctor in appropriate diagnosis, even when the patient is in a remote location. According to the on-line diagnosis system 500 including the diagnosis support apparatus 50 to which the fifth example embodiment is applied, it is possible to respond to the demand for the on-line diagnosis, because the doctor may be assisted in appropriate diagnosis, even when the patient is in a remote location.

The above describes an example in which the biometric information estimation apparatus according to the fifth example embodiment is applied to the on-line diagnosis system, but the biometric information estimation apparatus may be applied to other than a mechanism for medical purposes such as the on-line diagnosis system. For example, regardless of suffering from a disease or not, the target person would like to know his/her own physical condition. In order to respond to such a desire, for example, a dedicated application for estimating the biometric information may be installed in a terminal apparatus such as a smart phone carried by the target person. This dedicated application may support a series of operations, such as imaging the face image, transmitting the face image to a cloud as the biometric information estimation apparatus, receiving the biometric information such as heart rate and oxygen saturation from the cloud, and presenting the biometric information. This dedicated application may be available through the Internet. Furthermore, for example, the biometric information estimation apparatus may be mounted on a mobile terminal such as a smartphone carried by the target person. In this instance, the mobile terminal may include at least a camera, the biometric information estimation apparatus, and a display. For example, first, the target person may capture his/her own face image. Then, the biometric information estimation apparatus may acquire the face image to estimate at least one of the heart rate and the oxygen saturation of the target person, and may present at least one of the own heart rate and oxygen saturation to the target person through the display.

In addition, the fifth example embodiment describes an example in which the biometric information estimation apparatus acquires the face, but the image to be acquired may be an image of a skin at any part of the body, other than the face image, and may be, for example, a fingerprint image. Furthermore, the biometric information estimation apparatus may acquire biometric information on the target person other than the skin image, such as voice/speech data, heart rate, blood pressure level, heart rate, cardiac output, total peripheral resistance, pulse, oxygen saturation, electrocardiogram, body temperature, body moisture status, alcohol concentration, lactic acid level, blood sugar level, muscle activity, line-of-sight movement, brain activity, consciousness level, and stress level. In this instance, the terminal apparatus may be equipped with a detection apparatus such as an optical sensor and a potential biosensor, corresponding to the biometric information to be acquired. The biometric information estimation apparatus may be configured to estimate the biometric information such as heart rate, blood pressure level, cardiac output, total peripheral resistance, pulse, oxygen saturation, respiration rate, electrocardiogram, body temperature, body moisture status, alcohol concentration, lactic acid level, blood sugar level, muscle activity, line-of-sight movement, brain activity, consciousness level, and stress level.

### (6) Supplementary Notes

With respect to the example embodiment described above, the following Supplementary Notes are further disclosed.

### [Supplementary Note 1]

An information processing apparatus including:
an acquisition unit that acquires a first modal set that includes at least a first type of modal of multiple types of modals and that does not include a second type of modal of the multiple types of modals;
an output unit that allows a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and
a modal generation unit that generates a third modal set including the first modal set and the second type of modal outputted by the modal generation model.

### [Supplementary Note 2]

The information processing apparatus according to Supplementary Note 1, further including a first model generation unit that generates the modal generation model by performing the machine learning using the second modal set.

### [Supplementary Note 3]

The information processing apparatus according to Supplementary Note 1 or 2, wherein the output unit allows the modal generation model to output the second type of modal, by inputting, to the modal generation model, environmental information indicating an acquisition environment when the first type of modal is acquired.

### [Supplementary Note 4]

The Information processing apparatus according to Supplementary Note 3, wherein the modal generation model includes: an encoder unit that transforms features of at least one of the multiple into latent variables types of modals in a case where at least one of the multiple types of modals is inputted; and a decoder unit that generates at least one of the multiple types of modals by reconstructing the latent variables, and the output unit inputs the environmental information to at least one of the encoder unit and the decoder unit.

### [Supplementary Note 5]

The information processing apparatus according to any one of Supplementary Notes 1 to 4, further including a second model generation unit that generates a modal estimation model that outputs a fourth type of modal that is different from a third type of modal of the multiple types of modals as an output modal, in a case where the third type of modal of the multiple types of modals is inputted as an input modal, by performing machine learning using the third modal set.

### [Supplementary Note 6]

The information processing apparatus according to any one of Supplementary Notes 1 to 5, wherein the multiple types of modals are multiple types of biometric information including information about at least one of a face image, heart rate, and oxygen saturation.

### [Supplementary Note 7]

A biometric information estimation apparatus including:
an image acquisition unit that acquires a face image of a target person; and
a biometric information estimation unit that allows a modal estimation model to output biometric information on the target person as an output modal, wherein the modal estimation model outputs a fourth type of modal that is different from a third type of modal of the multiple types of modals as an output modal, in a case where the third type of modal of the multiple types of modals is inputted as an input modal, by performing machine learning using a third modal set including (i) a first modal set that includes at least a first type of modal of the multiple types of modals and that does not include a second type of modal of the multiple types of modals, and (ii) the second type of modal outputted by a modal generation model, wherein the modal generation model outputs at least one of the multiple types of modals in a case where at least one of the multiple types of modals is inputted, and the modal generation model is generated by machine learning using a second modal set including the multiple types of modals.

### [Supplementary Note 8]

The biometric information estimation apparatus according to Supplementary Note 7, wherein the image acquisition unit acquires the face image from an image generation apparatus that generates the face image by imaging the target person for whom the face image is generated, through a communication line.

### [Supplementary Note 9]

The biometric information estimation apparatus according to Supplementary Note 7 or 8, wherein the biometric information includes information about at least one of heart rate and oxygen saturation.

### [Supplementary Note 10]

An information processing method including:
acquiring a first modal set that includes at least a first type of modal of multiple types of modals and that does not include a second type of modal of the multiple types of modals;
allowing a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and generating a third modal set including the first modal set and the second type of modal outputted by the modal generation model.

### [Supplementary Note 11]

A recording medium on which a computer program that allows a computer to execute an information processing method is recorded, the information processing method including:
acquiring a first modal set that includes at least a first type of modal of multiple types of modals and that does not include a second type of modal of the multiple types of modals;
allowing a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and
generating a third modal set including the first modal set and the second type of modal outputted by the modal generation model.

At least a part of the constituent components of each of the example embodiments described above can be combined with at least another part of the constituent components of each of the example embodiments described above, as appropriate. A part of the constituent components of each of the example embodiments described above may not be used. Furthermore, to the extent permitted by law, all the references (e.g., publications) cited in this disclosure are incorporated by reference as a part of the description of this disclosure.

This disclosure is not limited to the examples described above and is allowed to be changed, if desired, without departing from the essence or spirit of this disclosure which can be read from the claims and the entire identification. An information processing apparatus, a biometric information estimation apparatus, an information processing method, and a recording medium with such changes are also intended to be within the technical scope of this disclosure.

### DESCRIPTION OF REFERENCE CODES

1, 2, 4 Information processing apparatus
11, 211 Acquisition unit
12, 212 Modal generation unit
13, 213 Modal output unit
IMS Partial modal set
GMS Generation complete modal set
1M First type of modal
2M Second type of modal
21, 51 Arithmetic apparatus
214 First model generation unit
22, 52 Storage apparatus
MMS Complete modal set
GM Modal generation model
GME Encoder unit
GMD Decoder unit
EL Environmental label
312 Input value generation unit
414 Second model generation unit
EM Modal estimation model
3M Third type of modal
4M Fourth type of modal
500 On-line diagnosis system
50 Diagnosis support apparatus
515 Image acquisition unit
516 Biometric information estimation unit
53 Communication apparatus
61 Image generation apparatus

What is claimed is:

1. An information processing apparatus comprising:
at least one memory that is configured to store instructions; and
at least one processor that is configured to execute the instructions to:
acquire a first modal set that includes at least a first type of modal of multiple types of modals and that does not include a second type of modal of the multiple types of modals;
allow a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and
generate a third modal set including the first modal set and the second type of modal outputted by the modal generation model.

2. The information processing apparatus according to claim 1, wherein the at least one processor is configured to execute the instructions to generate the modal generation model by performing the machine learning using the second modal set.

3. The information processing apparatus according to claim 1, wherein the at least one processor is configured to execute the instructions to allow the modal generation model to output the second type of modal, by inputting, to the modal generation model, environmental information indicating an acquisition environment when the first type of modal is acquired.

4. The Information processing apparatus according to claim 3, wherein
the modal generation model includes: an encoder unit that transforms features of at least one of the multiple types of modals into latent variables in a case where at least one of the multiple types of modals is inputted; and a decoder unit that generates at least one of the multiple types of modals by reconstructing the latent variables, and
the at least one processor is configured to execute the instructions to input the environmental information to at least one of the encoder unit and the decoder unit.

5. The information processing apparatus according to claim 1, wherein the at least one processor is configured to execute the instructions to generate a modal estimation model that outputs a fourth type of modal that is different from a third type of modal of the multiple types of modals as an output modal, in a case where the third type of modal of the multiple types of modals is inputted as an input modal, by performing machine learning using the third modal set.

6. The information processing apparatus according to claim 1, wherein the multiple types of modals are multiple types of biometric information including information about at least one of a face image, heart rate, and oxygen saturation.

7. A biometric information estimation apparatus comprising:
at least one memory that is configured to store instructions; and
at least one processor that is configured to execute the instructions to:
acquire a face image of a target person; and
allow a modal estimation model to output biometric information on the target person as an output modal, wherein the modal estimation model outputs a fourth type of modal that is different from a third type of modal of the multiple types of modals as an output modal, in a case where the third type of modal of the multiple types of modals is inputted as an input modal, by performing machine learning using a third modal set including (i) a first modal set that includes at least a first type of modal of the multiple types of modals and that does not include a second type of modal of the multiple types of modals, and (ii) the second type of modal outputted by a modal generation model, wherein
the modal generation model outputs at least one of the multiple types of modals in a case where at least one of the multiple types of modals is inputted, and the modal generation model is generated by machine learning using a second modal set including the multiple types of modals.

8. The biometric information estimation apparatus according to claim 7, wherein the at least one processor is configured to execute the instructions to acquire the face image from an image generation apparatus that generates the face image by imaging the target person for whom the face image is generated, through a communication line.

9. The biometric information estimation apparatus according to claim 7, wherein the biometric information includes information about at least one of heart rate and oxygen saturation.

10. An information processing method comprising:
acquiring a first modal set that includes at least a first type of modal of multiple types of modals and that does not include a second type of modal of the multiple types of modals;
allowing a modal generation model to output at least the second type of modal, by inputting the first modal set to the modal generation model, wherein the modal generation model outputs at least one of the multiple types of modals in a case where at least one of the multiple types of modals is inputted, and the modal generation model is generated by machine learning using a second modal set including the multiple types of modals; and
generating a third modal set including the first modal set and the second type of modal outputted by the modal generation model.

11. (canceled)
